(19) United States
(12) Patent Application Publication
(10) Pub. No.: US 2025/0249133 A1
(43) Pub. Date: Aug. 7, 2025

(54) RADIOEMBOLIC BEADS AND METHODS FOR TREATMENT OF TUMOR CELLS

(71) Applicant: NED Medical, Inc., Norwell, MA (US)

(72) Inventors: James Douglas Mitchell, Walnut Creek, CA (US); Andrew Allen Thoreson, Long Lake, MN (US); Richard Yazbeck, Norwell, MA (US)

(73) Assignee: NED Medical, Inc., Norwell, MA (US)

(21) Appl. No.: 18/852,768

(22) PCT Filed: Apr. 13, 2023
(86) PCT No.: PCT/US2023/018445

§ 371 Date: Sep. 30, 2024

Related U.S. Application Data
(60) Provisional application No. 63/337,773, filed on May 3, 2022.
(60) Provisional application No. 63/330,389, filed on Apr. 13, 2022.

(51) Int. Cl. A61K 51/12; A61K 9/16; A61K 51/06

(52) U.S. Cl. CPC A61K 51/1244; A61K 9/167; A61K 51/06

(57) ABSTRACT

Disclosed herein are methods, devices, and systems including embolic particles for use in treatment of tumor cells. The embolic particle can include an inner core and an outer layer. The inner core can have a volume within which a radioisotope can be accommodated, the inner core can include a surface over which a layer can be arranged. The outer layer can have a thickness within which a therapeutic can be accommodated, the outer layer can be placed over the surface of the inner core. The inner core and outer layer can have a combined density sufficient to allow the embolic particle to move with a fluid flow, along a pathway, while the embolic particle includes a dimension that is sufficiently large to engage the pathway to limit fluid flow to the tumor cells

### CROSS-REFERENCE TO RELATED APPLICATION

This patent application claims the benefit of U.S. Provisional Application No. 63/330,389, filed Apr. 13, 2022, entitled RADIO-EMBOLIC BEADS FOR TREATING CANCER, and the benefit of U.S. Provisional Application No. 63/337,773, filed May 3, 2022, entitled RADIO-EMBOLIC BEADS FOR TREATING CANCER, each of which is incorporated by reference in their entirety herein.

### TECHNICAL FIELD

The present disclosure is directed to injectable particles for the intravascular embolization of tumor cells, with or without the local delivery of radiation or therapeutics.

### BACKGROUND

Malignant tumors of the liver include primary tumors like hepatocellular carcinoma (HCC) and intrahepatic cholangiocarcinoma, with HCC being the most common primary liver tumor. Additionally, metastatic tumors from sites such as the bowel, breast, lung, and esophagus can involve the liver. For some patients, curative treatment can be offered through surgical resection of a liver tumor. However, many patients with primary or metastatic liver cancers will have underlying medical comorbidities or impaired liver function that preclude radical liver surgery. Additionally, the anatomic location or extent of a liver tumor may render it technically unresectable.

### SUMMARY

Alternative treatment modalities for inoperable patients with liver tumors includes selective intraarterial embolization of the tumor with microsphere particles. Liver tumors possess an alternate blood supply from the normal liver parenchyma. Most primary and metastatic liver tumors will receive the majority of their blood supply from the systemic arterial circulation through branches of the celiac axis. However, the normal hepatocytes receive their blood supply through the portal venous circulation. By taking advantage of this difference in blood supply, intraarterial embolization selectively targets the tumor vasculature while preserving the majority of the blood supply for normal hepatocytes. Further, similar to the liver, the lungs are supplied with blood from two distinct supplies from the pulmonary artery and the bronchial artery. Most lung tumors derive their blood supply from the bronchial artery while the majority of the lung parenchyma derives its blood supply from the pulmonary artery. Therefore, much the same as the liver, branches of the bronchial artery can be embolized with a low risk of damage to the surrounding normal lung parenchyma. Further still, the instant disclosure includes applicability in treatment of gliomas in the brain or spinal cord and tumors of the prostate, among others.

Microspheres used for embolization of liver tumors can include drug eluting materials that are used to deliver chemotherapeutic agents. Alternatively, microspheres may contain radioactivity for a procedure commonly known as radioembolization. The most commonly used radioisotope is yttrium-90 (Y-90), which is a pure beta emitting isotope. Y-90 has a half-life of 64.1 hours and the beta particle emitted has an energy of 2.28 MeV. Y-90 is produced through the decay of strontium-90, a fission product of uranium in nuclear reactors, and it decays to zirconium-90. There are currently only two available Y-90 microspheres. Currently available glass spheres are microspheres composed of glass measuring 20-30 microns. Currently available resin spheres are resin spheres measuring 20-60 microns.

Low linear energy transfer forms of radiation, including the 2.28 MeV beta particles produced through the decay of Y-90, kill cancer cells through what is known as the indirect effect. The indirect effect causes strand breaks in the phospho-ribose backbone of DNA in chromosomes. Single strand breaks are easily repaired, but double strand DNA breaks will often lead to cell death at the time of mitosis. The indirect effect is mediated by the formation of hydroxyl and peroxide free radicals that are created when ionizing radiation passes through the body. The formation of these free radicals requires the presence of oxygen, and therefore the indirect effect is enhanced in well oxygenated tissues. This can be mathematically represented as the oxygen enhancement ratio (OER) and tissues with a robust vascular supply of well oxygenated blood have a higher OER. Through embolization of the small arteries feeding a tumor, the cancer cells are in a more hypoxic environment leading to a lower OER and thus a lower level of tumor cell kill.

Radiosensitizers are chemicals that enhance radiation related cell kill. Hypoxic cell radiosensitizers selectively enhance the killing of hypoxic cells, while having little effect on cells with normal oxygenation. Nitroimidazoles are a class of antibiotic medications (metronidazole being the most widely used as an antimicrobial) that also provide hypoxic cell radiosensitization. Misonidazole is a second generation 2-nitroimidazole that was shown to improve outcomes when used in conjunction with radiotherapy to treat head and neck cancer patients in a Dutch randomized study (DAHANCA 2). More recently, DAHANCA 5-85 showed an improvement in both local control and overall survival with the addition of nimorazole to radiotherapy for treatment of head and neck cancer. Unfortunately, the systemic use of medications like misonidazole and nimorazole is limited by their side effects including central nervous system toxicity, and the logistics of accurately timing the medication dose with radiotherapy.

Local delivery of a hypoxic cell radiosensitizer could increase the therapeutic ratio of radioembolization by enhancing tumor cell kill while also mitigating the systemic side effects of the radiosensitizing drug. In 1992, Wang, et al, published the results of an animal study that involved intrahepatic arterial infusion of misonidazole in rabbits with VX2 liver cancer cells. The hepatic artery infusion of misonidazole was then followed by 15 Gray external beam radiation therapy. When compared to rabbits who did not receive misonidazole, those who underwent hepatic arterial infusion demonstrated the greatest tumor response showing extensive fibrosis and necrosis.

In addition to hypoxic cell radiosensitization, many other chemicals and pharmaceuticals have been shown to have radiosensitizing properties. Chemotherapy medications are often delivered concurrently with external beam radiation therapy, taking advantage of synergistic cell killing effects due to radiosensitization. Alkylating agents and antimetabolite chemotherapy drugs inhibit DNA repair pathways that cells utilize to repair sublethal damage from ionizing radiation. The accumulation of sublethal damage that is not properly repaired leads to enhanced cell death. Taxane chemotherapy agents and other microtubule inhibitors arrest the cell cycle in the G2-M phase interface, where cells are most sensitive to radiotherapy. Additionally, through radiation induced upregulation of antigen presenting cells (dendritic cells, etc.) and other proinflammatory effects, there is a well-documented synergy between ionizing radiation and immunotherapies used to upregulate immune targeting of cancer cells. These medications include anti-CTLA4 drugs, anti PD-1 and PDL-1 drugs/checkpoint inhibitors, chimeric antigen receptor T-cell (CAR-T) therapy, and other immunomodulating medications.

Here a novel embolic bead is disclosed that combines a radiation emitting embolic bead with a portion that is capable of containing, and delivering, drugs, including a radiosensitizer. The disclosure described herein takes advantage of the enhanced radiation cell killing of a radiosensitizer while minimizing the systemic effects of the drug. By combining both on a single embolic bead, they can be delivered together in a single injection, and their colocation is assured, maximizing combined efficacy.

The present disclosure is directed to an embolic particle for use in treatment of tumor cells. The embolic particle include an inner core having a volume within which a radioisotope can be accommodated, the inner core including a surface over which a layer can be arranged; an outer layer having a thickness within which a therapeutic can be accommodated, the outer layer being placed over the surface of the inner core; and the inner core and outer layer having a density sufficient to allow the embolic particle to move with a fluid flow, along a pathway, while the embolic particle includes a dimension that is sufficiently large to engage the pathway to limit fluid flow to the tumor cells.

In some embodiments, the dimension of the embolic particle can be sufficient to substantially block the fluid flow through the pathway to the tumor cells. The embolic particle can treat at least one of tumors of a liver, lungs, prostate, or gliomas in a brain or spinal cord.

In some embodiments, the therapeutic can be infused in the outer layer. The outer layer can release the therapeutic to the tumor cells while the embolic particle limits the fluid flow to the tumor cells. The embolic particle can treat at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

In some embodiments, the inner core can be embedded with a radioisotope. The inner core can emit radiation, while the embolic particle limits the fluid flow, to irradiate the tumor cells. The embolic particle can treat at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

In some embodiments the inner core can be embedded with a radioisotope and a therapeutic can be impregnated in the outer layer. The embolic particle can limit the fluid flow to the tumor cells, the inner core can emit radiation to kill the tumor cells, and the outer layer can release the therapeutic to treat remaining tumor cells. The radioisotope can be yttrium-90 (Y-90). The embolic particle can treat at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

In some embodiments, the inner core can be formed from at least one of polymer, ceramic, glass, a glass-ceramic composite. The inner core can be sintered with a porosity of about 10% to about 75%. The outer layer can form a uniform shape about the inner core. The outer layer can be formed from a polymer. The outer layer can be embedded with a radiopaque material. The embolic particle can have a substantially spherical shape. The embolic particle can have a size of about 15 micrometers-about 1000 micrometers. The embolic particle can include at least one additional layer, the at least one additional layer can be distinct from the inner core and outer layer.

In an embodiment a kit for treatment of tumor cells, is provided herein. The kit includes a plurality of embolic particles, each embolic particle including, an inner core having a volume within which a radioisotope can be accommodated, the inner core including a surface over which a layer can be arranged; an outer layer having a thickness within which a therapeutic can be accommodated, the outer layer being placed over the surface of the inner core; and the inner core and outer layer having a density sufficient to allow the embolic particle to move with a fluid flow, along a pathway, while the embolic particle includes a dimension that is sufficiently large to engage the pathway to limit fluid flow to the tumor cells; a liquid containing the therapeutic and into which the plurality of embolic particles can be soaked to permit the therapeutic to be infused in the outer layer; a vial to accommodate the liquid.

In some embodiments, the liquid can be disposed in the vial. The therapeutic can be infused into the outer layer of the plurality of embolic particles when the plurality of embolic particles are disposed in the liquid. The liquid can include saline. The plurality of embolic particles can expand in size when the plurality of embolic particles are disposed in the liquid. The plurality of embolic particles can remain the same size when the plurality of embolic particles are disposed in the liquid.

A method of manufacturing an embolic particle, is disclosed herein. The method includes, providing an inner core having a volume within which a radioisotope can be accommodated, the inner core including a surface over which a layer can be arranged; providing a composition within which a therapeutic can be infused, and arranging the composition over the surface of the inner core to create an outer layer.

In some embodiments, the inner core can include a hollow inner chamber which can accommodate the radioisotope or the therapeutic. The method can additionally include infusing the radioisotope into the inner core. The method can further include, activating the radioisotope such that the embolic particle emits radiation. The method can further include, infusing the outer layer with the therapeutic. The therapeutic can be premixed with the outer layer prior to the arranging step.

A method of treatment of tumor cells using embolic particles is disclosed herein. The method includes, providing a plurality of embolic particles each having an inner core having a volume within which a radioisotope can be accommodated and an outer layer having a thickness within which a therapeutic can be accommodated; identifying a site of interest that includes the tumor cells to be treated; and delivering the embolic particles to a lumen and allowing the plurality of embolic particles to move with a fluid flow, along a pathway, to the site of interest such that the embolic particles engage with the pathway to limit fluid flow to the tumor cells.

In some embodiments, the method can further include activating radioisotopes within the inner core. The method can further include irradiating the tumor cells at the site of interest. In some embodiments, the method can further include impregnating the therapeutic into respective outer layers of the plurality of embolic particles. The impregnating step can further include disposing the plurality of embolic particles into a fluid containing the therapeutic to infuse the therapeutic into at least the outer layer. A dimension of respective embolic particles can limit movement of the respective embolic particles through the lumen at the site of interest to prevent the plurality of embolic particles from migrating away from the site of interest. The delivering step can include delivering the plurality of embolic particles through an arterial branch that feeds the tumor cells. The method can further include, treating at least one of tumors of a liver, lungs, prostate, or gliomas in a brain or spinal cord with the plurality of embolic particles.

### BRIEF DESCRIPTION OF THE DRAWINGS

FIG. 1A is a perspective, partially broken away, view of an embolic bead according to an embodiment of the present disclosure.

FIG. 1B is a perspective, partially broken away, view of an embolic bead having an inner core with radioisotope particles surrounded by an external layer with radiosensitizing medication.

FIG. 1C is a cross-sectional view of an embolic bead according to an embodiment of the present disclosure.

FIG. 2A is a perspective, partially broken away, view of an embolic bead composed of a single uniform sphere according to an embodiment of the present disclosure.

FIG. 2B is a perspective, partially broken away, view of an embolic bead having a single uniform sphere containing both radioisotope and radiosensitizing medication

FIGS. 3A-3C are cross-sectional views of embolic beads according to embodiments of the present disclosure.

FIGS. 4A and 4B are illustrations of methods of infusion according to embodiments of the present disclosure.

FIG. 5 is a cross-sectional view of an embolic bead according to an embodiment of the present disclosure.

FIG. 6 illustrates a method of infusion according to an embodiment of the present disclosure.

FIGS. 7A and 7B are illustrations of methods of infusion according to embodiments of the present disclosure.

### DETAILED DESCRIPTION

The present disclosure overcomes shortcomings of the aforementioned current devices by providing, in one embodiment, an embolic bead or particle having coadministration of a radioembolic and a therapeutic, e.g., a radiosensitizing agent. The instant embolic bead initially allows for an increase in tumor cell death from irradiation of a tumor. The instant embolic bead combines a source of radiation with a drug eluting component that contains a therapeutic, e.g., a radiosensitizer, in some embodiments, allowing for administration of the radiation therapy concurrently with, or followed by, the administration of the radiosensitizer. In some embodiments, the instant embolic bead allows for simultaneous administration of both therapeutics in the same location of the tumor microvasculature. The delivery of the drug from the outer layer can be dependent on the formulation of the outer layer to allow either immediate release of the drug upon delivery (by dissolving of the outer layer) or delayed/timed release of the drug (slower dissolving of the outer layer). Advantageously, the instant embolic beads allow for a combined modality therapy for interventional oncology while minimizing the radioresistance of tumors cells due to hypoxia or other cellular and molecular processes, providing for optimized activity, and having favorable flow characteristics. Such devices allow for curative treatments for various tumors, e.g., liver tumors, in an outpatient setting.

Said another way, the instant embolic beads, in the various embodiments, can advantageously provide for at least a dual treatment of certain tumor cells. By providing a single particle type having a source of radiation for radiation treatment, for example of cancers of the liver, as well as a drug eluting component containing a therapeutic, the instant embolic beads provide for a treatment that is more effective than one of the therapies alone. The instant embolic beads provide an important benefit that is not found in prior art treatments. Namely, prior art treatments that use only one of radiation or radiosensitizer, in contrast the instant disclosure provides for a more comprehensive treatment than previously provided for.

In general, cancers of the liver can be treated with embolization. Embolization can be used with tumors that cannot be removed by traditional surgery. For example, embolization can be used in cases where tumors are too large to be treated with ablation, e.g., tumors larger than 5 cm across, and who also have adequate liver function. Embolization can reduce the blood supply to the normal liver tissue, so it may not be a good option for some patients whose liver has been damaged by diseases such as hepatitis or cirrhosis. However, because the liver has two blood supplies, the hepatic artery, which often provides a blood supply to the cancer in the liver, can be blocked, or otherwise limited, while allowing the portal vein to supply blood to the healthy liver cells. Further, similar to the liver, the lungs are supplied with blood from two distinct supplies from the pulmonary artery and the bronchial artery. Just like liver tumors which attach themselves to the hepatic artery, almost all lung tumors are attached to the bronchial artery. Therefore, much the same as the liver, one can embolize branches of the bronchial artery without damaging the remaining healthy tissue in the lungs. Other organs and anatomic sites in the human body are amenable to implantation of radioactive sources directly into the interstitial tissues or within a body cavity, a procedure known as interstitial or intracavitary brachytherapy. The instant embolic beads can additionally be utilized for interstitial, or intracavitary brachytherapy, providing a means of anatomically localized concurrent delivery of therapeutic radiation and a therapeutic, e.g., a radiosensitizing agent, through a route other than transarterial embolization. As such, the instant disclosure includes applicability in treatment of primary or metastatic tumors in the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, spine, kidneys, ureters, bladder, urethra, vagina, uterus, cervix, ovary, lymph nodes, muscles, and bones, among others.

The embolic bead, in accordance with an embodiment of the present disclosure, includes a radioembolic or radiotherapy portion and a drug, or radiosensitizer, eluting portion. The radioembolic portion, in one embodiment, can be an inner portion of the embolic, and can be constructed from a substantially hard material, such as a polymer, glass, ceramic, or a glass-ceramic composite. To provide the inner portion with radiotherapy ability, the inner portion, in an embodiment, can be infused or embedded with Y-90 or a resin containing Y-90. Alternatively, any similar radioactive isotope could be utilized as the source of radiotherapy. As such, a substantially hard particulate material similar to glass or resin could also be used as a substrate for the radioisotope. In some embodiments, the radiation emitting portion can be a uniform mixture of glass, polymeric or hydrogel, ceramic, a glass-ceramic composite, or any other material with the radiation emitting radioisotope. Alternatively, the radiation emitting portion can be a non-uniform mixture of glass, polymeric or hydrogel, ceramic, or glass-ceramic composite, or any other material with the radiation emitting radioisotope.

In some embodiments, the radioembolic bead can be coated, encapsulated, or otherwise combined with, a drug eluting compound that can carry a therapeutic, or medication, for slow release after, or during, the period of irradiation. For example, in an embodiment, the therapeutic can be released after the period of irradiation to ensure that any tumor cells not killed by the irradiation are eliminated. Drug eluting embolics can include poly-vinyl-alcohol (PVA) polymers that are doped with sulfonyl groups allowing a static charge to bind with polarized molecules. Examples include DC/LC beads and QuadraSphere beads. Additional embolic beads used for chemoembolization can include Lipiodol, gelatin sponge, polymethylmethacrylate (Oncozene), and degradable starch (Spherex). However, it should be understood that these embolic beads do not share the same drug eluting properties as the PVA polymers.

In an embodiment, the radioembolic bead can include a radioembolic core of glass, ceramic, or resin containing Y-90 that can be coated with a drug eluting shell such as a PVA polymer, or other polymers. It is in the scope and spirit of the present disclosure to include other drug-eluting coatings, known or unknown, or other ways of incorporating drug within, or on the surface of, the particulate. For example, the PVA polymer can carry a hypoxic cell radiosensitizer for slow elution into the irradiated area.

In some embodiments, the embolic beads 1 of the present disclosure can be larger than about 15 micrometers in diameter to prevent passage though small arterial-venous shunts and non-target organ embolization, biliary ischemia, hepatobiliary infarction, or other untoward effects. In an embodiment, the embolic beads can include an upper limit size threshold of 1,000 micrometers, as larger sizes can cause occlusion of commonly used microcatheters. Additionally, large particles create a more proximal arterial occlusion and promote hypoxia and may sub-select more resistant cancer cell populations. However, in practice the embolization particles could be any size that fulfills the clinical requirement of reaching the desired anatomic distribution.

The present disclosure will now be described in detail hereinafter by reference to the accompanying drawings. The disclosure is not intended to be limited to the embodiments described; rather, this detailed description is provided to enable any person skilled in the art to make and practice the instant disclosure.

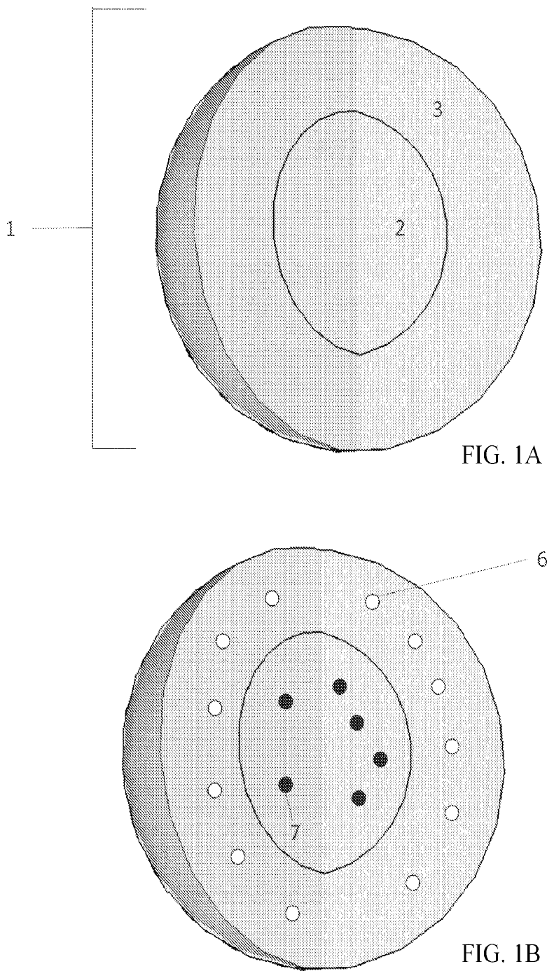

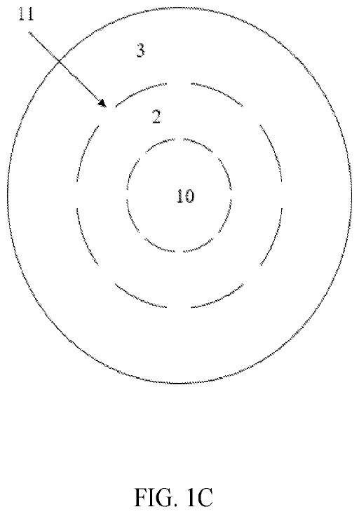

Turning to FIGS. 1A and 1B, the embolic bead 1, in an embodiment, can be comprised of an inner core 2 and outer layer 3. The embolic bead 1 can be anywhere from about 5 microns to about 1000 microns in overall size. The term size, or dimension, as used herein, can mean an outer dimension including width, length, diameter, etc. The embolic bead 1 can be any regular or irregular 3D shape including, but not limited to, spherical, half spherical, cubical, conical, cylindrical, octahedral, etc. The drawings presented in this disclosure are shown as spherical or circular. These are purely for illustrative purposes only and do not reflect the only embodiments that the particles can be. In some embodiments, at least a portion of the embolic bead 1 can have a radiopaque material embedded therein. The radiopaque material can, advantageously, allow for visualization of the delivery of one, or more, embolic beads 1 within a patient to a site of interest, e.g., to the location of a tumor.

The inner core 2, in accordance with one embodiment, can be made from a polymer, glass, ceramic, glass-ceramic composite, resin, or a combination thereof. It is also within the scope of this disclosure to have the core comprised of any other biocompatible material capable of housing a radioisotope. The inner core 2 of embolic bead 1, in an embodiment, can be provided with a volume within which can be disposed a radioisotope 7 capable of delivering local radiation therapy to the surrounding tumor when the embolic bead 1 is deployed to the tumor site. An example radioisotope used in such procedures can be yttrium-90 (Y-90) or Holmium-166, though it is within the scope of this disclosure to utilize any beta or gamma-emitting radioisotope known or unknown. In some embodiments, as shown in FIGS. 1A and 1B, the inner core 2 can be solid, e.g., a solid sphere. Alternatively, as shown in FIG. 1C, the inner core 2 can be porous, having a number of irregularly or regularly located pores 11, and include a hollow inner section 10 for containing additional medications or radiosensitizing compounds, including those described above. In some cases, the inner core 2 can be a solid or hollow core that is sintered to about 10-75% porosity. Depositing of a fluid in the hollow inner section 10 can be accomplished by immersing the embolic bead 1 into a bath of a specified fluid and allowing the fluid to deposit inside the particle, for example through pores 11. The particles can then be filtered out to separate them from the fluid bath. The filtration of the embolic bead 1 from the fluid bath can be done prior to or during the infusion of the embolic bead 1. The inner core 2 can, in some embodiments, be coated with at least one outer layer, such as the outer layer 3.

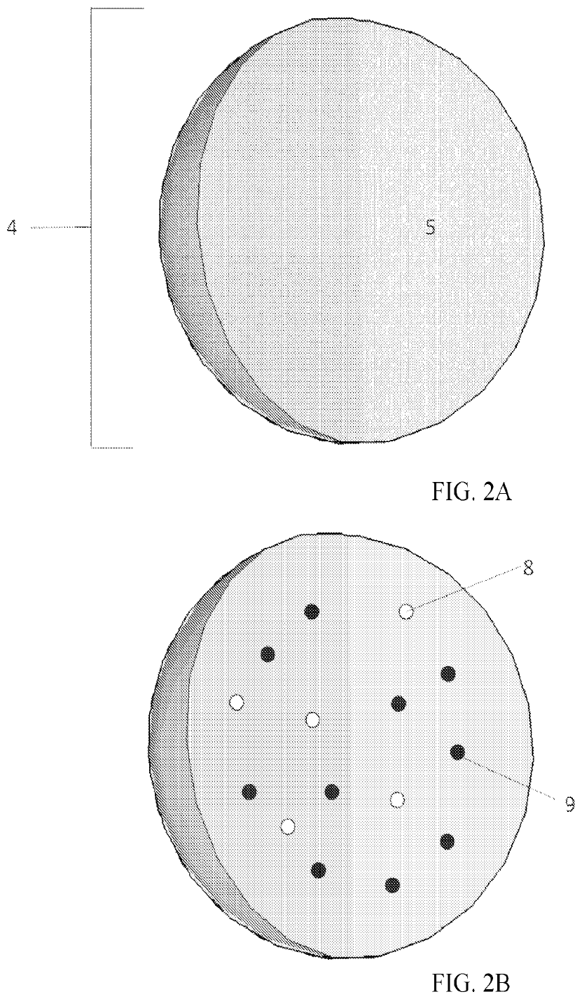

The outer layer 3, in an embodiment, can include a drug eluting, or releasing, glass, ceramic, ceramic/glass, resin, polymer, metal, or other biocompatible material capable of being deposited, adhered, coated, or otherwise attached to the inner core 2 and capable of drug delivery and elution when deployed in the body. In an embodiment, the outer layer 3 can be deposited, or coated, onto the inner core 2 such that the resulting embolic bead 1 does not have any rough or jagged edges to prevent the embolic bead 1 from damaging any healthy tissue it comes into contact with during the administration. In some embodiments, the outer layer 3 of embolic bead 1 can have a thickness, and therefore a resulting volume, which can accommodate a therapeutic, e.g., a radiosensitizing compound, hypoxic cell cytotoxins, immunotherapy, CAR-T therapies, etc., that can be diffused, or emitted, from the embolic bead into the surrounding tumor and body tissues at the same time, or a different time, as the radioisotope compound(s) are treating the tumor. In an embodiment, this compound can be a hypoxic cell radiosensitizer such as a nitroimidazole. However, it is within the scope of this disclosure to use any therapeutic compound, known or unknown. In some embodiments, the outer core 3 can remain intact after delivery to a site of interest within a patient, or alternatively, the outer core 3 can be absorbed into the body after delivery. Additionally, although it is shown in the figures to have the radioisotope in the core 2 and the drug eluting portion in the outer layer 3, the position of the two layers could be reversed to have the drug eluting portion in the core and the radioisotope in the outer layer. In this configuration, that materials that make up the core and outer layer could also need to be reversed. Alternatively, as shown in FIGS. 2A and 2B, in some embodiments, the embolic bead 4 can be a uniform material 5 such as a resin, glass, polymer, or ceramic-glass composite that forms the entirety of the bead. Intermixed within the embolic bead 4 can be radioisotopes 9 and radiosensitizers 8.

In some embodiments the therapeutic can be a radiosensitizer which can be nitroimidazole hypoxic cell radiosensitizer. Alternatively, the drug eluting portion could contain any other therapeutic compound including but not limited to non-nitroimidazole hypoxic cell radiosensitizers, radiosensitizing chemotherapeutics like taxanes (e.g., paclitaxel) or platinum containing compounds (e.g., cisplatin), or other radiosensitizing compounds yet to be identified.

In some embodiments, the instant embolic bead 1 can include the inner core 2 and the outer layer 3, where the inner core 2 does not contain radioactive material and the outer layer does not include a therapeutic. In such a case, the embolic bead 1 can perform TAE, or bland embolization of the tumor to starve the tumor of an energy source. Alternatively, in some cases, the instant embolic bead 1 can be used in trans-arterial chemoembolization (TACE). TACE is often the initial type of embolization used for large liver cancers that cannot be treated with surgery or ablation and combines embolization with chemotherapy (chemo). For example, the drug eluting portion can include Doxorubicin, Cisplatin, Epirubicin, Miriplatin, Carboplatin, Mitomycin C, Gemcitabine, or 5 FU. In the case of treating metastasized tumors originating from hepatocellular carcinoma (HCC), the drug eluting portion can include certain systemic agents including Atezolizumab, Bevacizumab, Tremelimumab-actl, Darvalumab, Soreafenib, Lenvatinib, Pembrolizumab, Nivolumab, Ipilimumab, Regorafenib, Cabozantinib, Ramucirumab, Dostarlimab, or Selpercatinib. In some embodiments, in the case of treating metastasized tumors originating from colorectal cancer, the drug eluting portion can include 5 FU, Oxaliplatin, Leukovorin, Capecitabine, Irinotecan, Bevavcizumab, Panitumumab, Nivolumab, Ipilimumab, Pembrolizumab, Trastuzumab, Pertuzumab, Lapatinib, Tucatinib, Ramucirumab, Ziv-aflibercept, Cetuximab, Panitumumab, Encorafenib, Dostarlimab-gxly, Lapatinib, Fam-traztuzumab deruxtecan, Regorafenib, Trifluridine, or Tipiracil.

In some embodiments, when treating metastasized tumors originating from cholanigiocarcinoma, the drug eluting portion can include those medications commonly treated with TACE including Doxorubicin, Cisplatin, Epirubicin, Miriplatin, Carboplatin, Mitomycin C, Gemcitabine, or 5 FU. Alternatively, the drug eluting portion can include any of the following 5FU, Capecitabine, Oxaliplatin, Leukovorin, Gemcitabine, Cisplatin, Durvalumab, Paclitaxel & NAB-paclitaxel, Regorafenib, Irinotecan, Lenvatinib, Pembrolizumab, Entrectinib, Larotrectinib, Nivolumab, Ipilimumab, Pralsetinib, Selpercatinib, Dostarlimab-gxly, Dabrafenib, Trametinib, Futibatinib, Pemigatinib, Ivosidenib, Traztuzumab, or Pertuzumab.

In some embodiments, when treating metastasized tumors originating from breast cancer, the drug eluting portion can include Adriamycin, Cyclophosphamide, Paclitaxel, Docetaxel, Olaparib, Pebrolizumab, Carboplatin, Epirubicin, Methotrexate, 5FU, Capecitabine, Trastuzumab, Pertuzumab, Neratinib, TDM-1, Tamoxifen, Anastrozole, Letrozole, Ribociclib, Abemaciclib, Palbociclib, Fulvestrant, Exemestane, or Everolimus. In some embodiments, when treating metastasized tumors originating from non-small cell lung cancer, the drug eluting portion can include Carboplatin, Paclitaxel, Cisplatin, Pemetrexed, Gemcitabine, Docetaxel, Vinorelbine, Etoposide, Nivolumab, Osimertinib, Atezolizumab, Pembrolizumab, or Darvalumab. In some embodiments, when treating metastasized tumors originating from prostate cancer, the drug eluting portion can include Nilutamide, Flutamide, Bicalutamide, Abiraterone, Enzalutamide, Apalutamide, Darolutamide, Docetaxel, Ketoconazole, Cabazitaxel, Carboplatin, Mitoxantrone, Pembrolizumab. In some embodiments, when treating metastasized tumors originating from pancreatic cancer, the drug eluting portion can include 5FU, Oxaliplatin, Irinotecan, Leucovorin, Gemcitabine, Paclitaxel, Nab-paclitaxel, Cisplatin, Erlotinib, Dabrasfenib, Trametinib, Pembrolizumab, Larotrectinib, Entrectinib, Dabrafenib, Olaparib, Rucaparib.

By combining the radioisotope and a drug eluting layer infused with a therapeutic on the same embolic bead, or particulate, the tumor cell kill can be enhanced while maintaining a simplified procedure requiring only a single hepatic artery cannulation and a single injection of therapeutic material. Additionally, the range of the Y-90 emitted beta particles is on the order of only 1 mm in tissue. It is therefore critical to ensure colocation of the radiation source and the radiosensitizer elution. This can be accomplished through coadministration on the same embolic bead, as combining a radioembolic bead with separate drug eluting particles would not guarantee that both therapeutics would be delivered to the same anatomic location. In some embodiments, the instant embolic beads can be used for TAE (or bland embolization), TACE (or chemoembolization), and TARE (or radioembolization), in addition to bio-radioembolization which involves concurrent delivery of therapeutics. In the case of TARE, the instant embolic bead can have the capability to deliver drugs if the physician so choses to do. In some embodiments in the case of TARE, the instant embolic bead 1 can have the radioactive material that is embedded within the inner core 2 activated, without a therapeutic embedded within the outer layer 3. In such an embodiment, the embolic bead 1 can be infused into the patient to allow the embolic bead 1 to treat a tumor at a site of interest by emitting radiation alone. In some embodiments, the therapeutic material can be infused into the outer layer 3 after the time of delivery to a hospital, or healthcare facility, but before infusion to the patient. In some embodiments, the instant embolic bead can provide for the specific combination of a particular drug with the radiation-emitting beads. In some cases, the instant embolic beads may not have activated radioisotopes, which would result in a particle with a core and a polymeric coating on the outside, which would also serve the TAE and TACE markets. Therefore, the instant embolic beads allow for embolic beads that can have radiation emission, drug delivery and embolization capability while some embolic beads can only have the drug delivery and embolization (no radiation).

In some embodiments, the radiosensitizer can be used to treat a number of metastasized tumors effecting the liver. For example, the radiosensitizer can be nitroimidazole hypoxic cell radiosensitizer. Alternatively, the drug eluting portion could contain any other radiosensitizing compound including but not limited to non-nitroimidazole hypoxic cell radiosensitizers, radiosensitizing chemotherapeutics like taxanes (e.g., paclitaxel) or platinum containing compounds (e.g., cisplatin), or other radiosensitizing compounds yet to be identified. For example, in some embodiments, in the case of treating metastasized tumors in the liver originating from colorectal cancer, the drug eluting portion, or outer layer 3, can be infused with 5 FU, Oxaliplatin, Leukovorin, Capecitabine, Irinotecan, Bevavcizumab, Panitumumab, Nivolumab, Ipilimumab, Pembrolizumab, Trastuzumab, Pertuzumab, Lapatinib, Tucatinib, Ramucirumab, Ziv-aflibercept, Cetuximab, Panitumumab, Encorafenib, Dostarlimab-gxly, Lapatinib, Fam-traztuzumab deruxtecan, Regorafenib, Trifluridine, or Tipiracil.

In some embodiments, the inner core 2 can have a first material density and the outer layer 3 can have a second material density. The first material density can be, in some embodiments, denser than the second material density. However, advantageously, the embolic bead 1 made of the inner core 2 and the outer layer 3 can have a lower overall, or combined, density than the inner core 2 alone such that the embolic bead 1 can be buoyant, or neutrally buoyant, within a fluid therefore the embolic bead 1 can have a favorable flow characteristic within the fluid, e.g., human blood. A favorable flow characteristic can be understood to mean that the embolic bead 1 can flow within the fluid without sinking and becoming lodged within a lumen, e.g., a human artery or vein. The lower density of the embolic bead 1 can be a function of the ratio of volumes of the inner core 2 to the outer layer 3. In some embodiments, the lower density can also be a function of the physical characteristics of the inner core 2. For example, an inner core 2 with pores or a hollow cavity may be more buoyant than a solid inner core of the same dimensions.

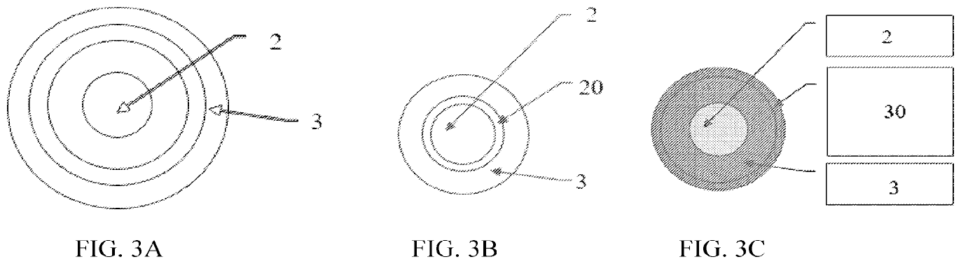

In some embodiments, the embolic bead 1 can be constructed with any combination of layers such as, an inner core 2 of radiation emitting material 7 and an outer layer 3 of a drug-eluting material 6, as shown in FIGS. 1A and 1B. Alternatively, the embolic bead 1 can be formed from any plurality of layers, as shown in FIG. 3A, with some combination of one of the layers being radiation emitting 2 and one of the layers being drug eluting 3. In some embodiments, as shown in FIG. 3B, the embolic bead 1 may incorporate a binding layer 20 that bonds the drug-eluting layer 3 to the other layers, such as the radiation emitting layer 2. The binding layer 20 could be incorporated at the interface of any of the various layers. The binding layer can be a binding agent that can be polymeric in nature or utilizes some form of ionic bonding. The embolic bead 1 may incorporate a sacrificial encapsulation layer 30 that contains the eluting drug with respect to the rest of the particle, as seen in FIG. 3C. The sacrificial layer can dissolve and go away upon administration of the particle to the patient, exposing the drug elution layer 3 at the desired location of interest.

In a method of use, there may be a need for activation of drug elution. For example, to initiate the delivery of a drug at a site of interest, can include the elimination of a sacrificial layer 30 to expose the drug eluting layer 3, as shown in FIG. 3C, or activating an already exposed layer of the drug. The activation can be accomplished in numerous ways including, exposure of the embolic bead 1 to body fluid; exposure of the embolic bead 1 to body heat; exposure of the embolic bead to an external energy source such as radiation, heat, MRI, or ultrasound; exposure of the embolic bead 1 to fluids, such as saline, external to the patient, prior to administration of the particles; exposure of the embolic bead 1 to light; exposure of the embolic bead 1 to magnetism; injecting CO2 that can react with the drug; and/or radiation emission from the isotope cleaving within the core 2 to convert the medication. In some embodiments, the method can include percutaneous delivery of an energy delivery device like ablation/heat to the site of interest, after the delivery of the embolic beads 1, and delivering energy with the energy delivery device like ablation/heat at the site of interest. In an embodiment, the method of activating the medication can include the use of an internal gas source within the implanted particle that expands and ruptures or develops a hole upon exposure to body temperature or some other energy source.

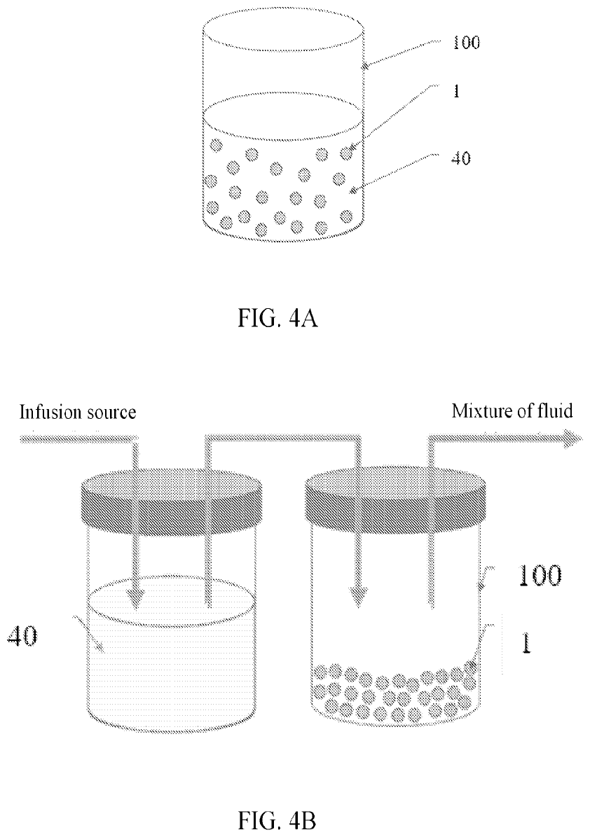

In some embodiments, a method for delivering a radiation emitting, micron-sized, embolic bead 1 that can be mixed into a fluid 40, is provided. For example, the embolic bead 1 and the fluid 40 can be infused into the patient simultaneously, as shown in FIG. 4A. In some embodiments the fluid 40 can be a chemotherapeutic drug, a hypoxic radiosensitizer drug, or any other agent used to treat tumor cells. The term radiosensitizer used herein can refer to hypoxic cell radiosensitizers, bioreductive drugs, radiosensitizing chemotherapy agents, immunotherapies, or any other radiosensitizing chemical, protein, medication, or compound known today or in the future. The embolic beads 1 and fluid 40 can be contained in a single vial 100 and delivered as such to a clinician for use. For example, the embolic bead 1 can be shipped where the drug eluting layer 3 does not include any therapeutic agents. In such a case, the hospital can mix the embolic bead 1 with a therapeutic agent, via fluid 40, before administering the embolic beads 1 to a patient.

In some embodiments, the embolic bead 1 may need the fluid 40 to “activate” the drug eluting layer 3, which can be a polymeric layer. In the case of a polymeric later, or coating, the fluid 40 can be approximately 100% NaCl 0.9% aqueous solution, non-ionic contrast medium, or approximately a 50/50 mix of NaCl 0.9% aqueous solution and contrast. Alternatively, the fluid 40 can be any of the therapeutic agents disclosed herein. As the polymeric layer, e.g., the drug eluting layer 3, is exposed to the fluid 40, the embolic beads 1 can swell such that they expand in diameter. Alternatively, the exposure of the fluid 40 to the embolic beads may activate the drug eluting layer 3 without causing the embolic beads 1 to swell. In some embodiments, the introduction of the embolic beads 1 to the fluid 40 can occur before, or after, the embolic beads 1 are shipped to the hospital. In an alternative embodiment, the radiation emitting, micron-sized, embolic beads 1 delivered in a separate vial 100 from the fluid 40 with the two mixed and infused via a delivery mechanism, as shown in FIG. 4B.

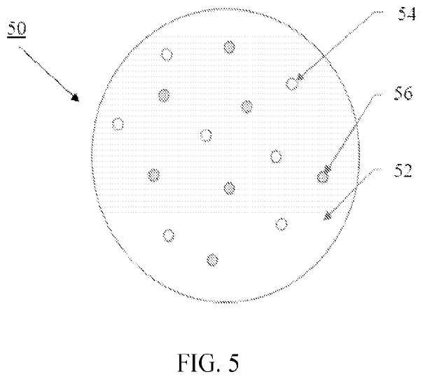

In some embodiments, the embolic beads can be a radiation emitting, drug-eluting, embolic beads. In comparison to the embolic bead 1 of FIGS. 1A and 1B, for example, the particle 50 can be a single mixture of a base material 52, a radiation-emitting isotope 54, and drug medication 56, as seen in FIG. 5. As the base material is absorbed in the body, the drug medication can be time released to advantageously allow for the medication 56 and radiation-emitting isotope 54 to be provided to the patient in larger quantities without causing detrimental side effects. The particle 50 can be sized from about 5 microns to about 1000 microns in overall size. The particle 50 can be made from any material that is bioresorbable such as a polymer, hydrogel, etc. The particle 50 can be any regular, or irregular, 3D shape including spherical, half spherical, cubical, conical, cylindrical, octahedral, etc. While the drawings presented in this disclosure show the particle 50 as being spherical or circular, these figures are purely for illustrative purposes only and do not reflect the only embodiments that the particles can be.

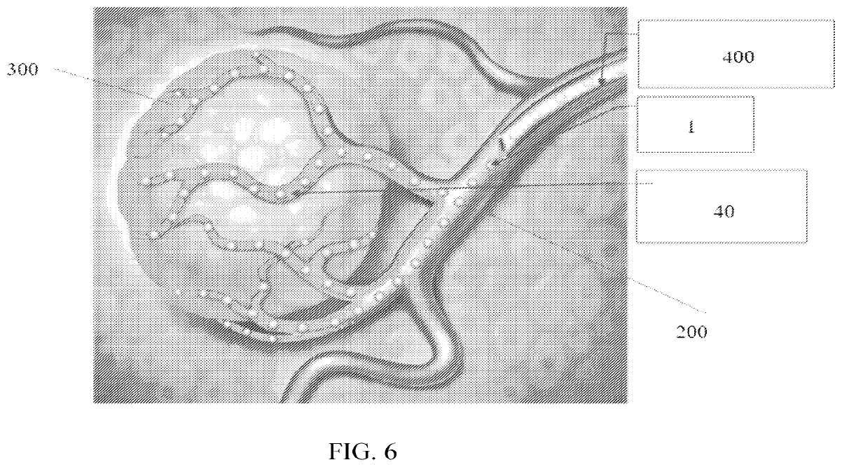

In a method of delivery of the instant embolic beads, according to an embodiment, the method can encompass the non-concurrent delivery of a radiation emitting, embolic, particle 1 and a fluid 40 (e.g., chemotherapeutic drugs, hypoxic radiosensitizer drugs, or any other agent used to treat tumor cells), as shown in FIG. 6. In some embodiments, the fluid 40 can first be delivered to the arterial branch 200 that feeds the tumor 300 by a microcatheter 400. Either immediately, or later, the method can include the delivery of the radioembolic beads 1 into the same arterial branch 200. The idea is that the fluid 40, or drugs, are delivered to the tumor 300 and the radioembolic beads 1 can occlude the lumen to capture the drugs in place and prevent systemic migration of the drugs. In addition, and advantageously, the radioembolic bead 1 can additionally deliver the necessary radiation to treat the tumor 300. Alternatively, the method can include delivering the drug directly into the tumor using an ultrasound-guided, percutaneous method followed by delivery of the radioembolic using a standard technique of a microcatheter placed in the appropriate vessel.

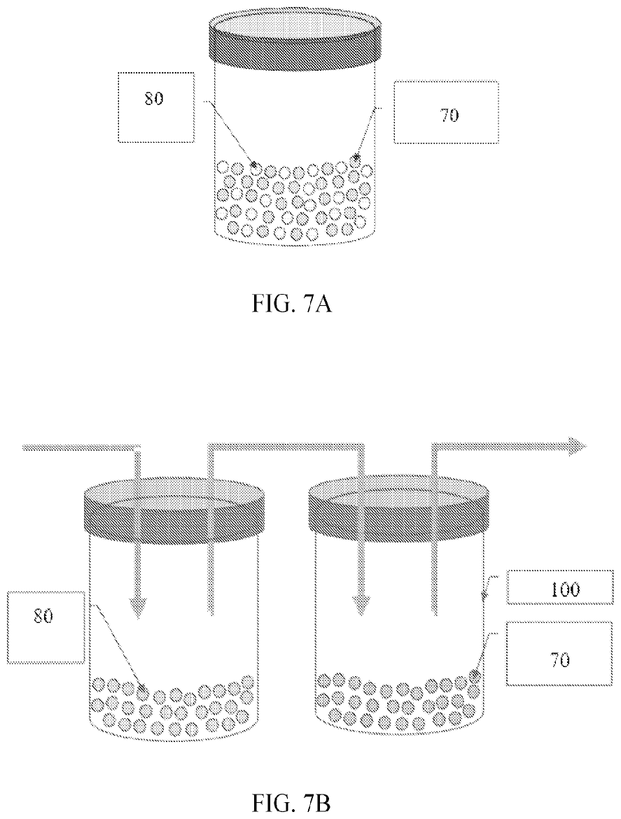

Some embodiments can include combining two separate particles. One particle 70 can be a radiation emitting, micron-sized, embolic bead and the second particle 80 can be micron-sized and drug eluting. Eluting of the drugs can be accomplished in any of the ways described in the above embodiments. In some embodiments, the drug can be a chemotherapeutic drug, a hypoxic radiosensitizer drug, or any other agent used to treat tumor cells, for example those disclosed herein. The particles 70, 80 can be any shape or material as described in other embodiments above. The two particles 70, 80 can be the same material or different materials and can be the same or different densities. In some embodiments, the two particles 70, 80 can be the same size and shape, or, alternatively, they can be two distinct sizes and shapes. The two particles 70, 80 can come already mixed together as shown in FIG. 7A or can be mixed at the time of infusion into the patient via a delivery mechanism, as shown in FIG. 7B. Alternatively, in some embodiments, the delivery method can involve separately infusing the drug eluting particles 80 first into the patient and then infusing the radiation emitting particles 70 behind it, or vice versa. The ratio of one particle to the other can be a fairly broad range.

In some embodiments, in addition to the drugs that are added to the embolic bead 1, there can be the addition of a CAR-T therapy, or other cellular therapies, as an additional element. Cellular therapies, including CAR-T, typically require extraction of a patient's native cells, genetic modification of those cells, and reintroduction of the modified cells into the patient. In the case of CAR-T, a patient's T cells (both CD4 and CD8) can be harvested from a patient. The T cells are then genetically engineered to recognize and target tumor specific antigens through a gene editing technology such as CRISPR/Cas9. The modified T cells can then be reintroduced into the patient through infusion, stimulating an immune response targeting cancer cells. CAR-T can not only include targeting specific antigen and proteins but can also be tailored to target hypoxic cancer cells. This is different from hypoxic cell radiosensitization, in that CAR-T can be more like bioreductive drugs, which are directly toxic to hypoxic cells. Therefore, the CAR-T therapy could be introduced in addition to the hypoxic cell radiosensitizer drug, not necessarily replace it. Systemic administration of CAR-T therapy frequently leads to Cytokine Release Syndrome (CRS), a potentially life-threatening inflammatory reaction to treatment. It is possible that localized delivery of CAR-T cells directly to a tumor, for example with the instant embolic beads, can mitigate the severity of CRS.

The introduction of CAR-T into embolic bead 1, can create a radioembolic with the following modalities. The embolic bead 1, in an embodiment, can provide for 1) vascular embolization to stop the flow of blood to the tumor cells; 2) radiation emission to expose the tumor cells to ionizing radiation, killing tumor cells and upregulating tumor related antigen presentation; 3) radiosensitization to enhance radiation induced cell death in the tumor; and 4) CAR-T to target the tumor cells over a prolonged period of time after the radiation emission and hypoxic cell radiosensitizer have subsided.

Multiple embodiments of the invention disclosed herein may be used to deliver cellular therapy including CAR-T. For example, in one embodiment, a hollow particle method as described in FIG. 1C may be used, where the hollow spheres are immersed in the CAR-T therapy and then subsequently filtered out and injected into the patient or injected simultaneously with the particle. In some embodiments, one can also use the method described in FIG. 3A, where the radioembolic bead 1 could be constructed in layers with each layer constituting one specific modality (radiation emission, drug elution, CAR-T delivery). Alternatively, one can use the method described with respect to FIG. 6, where the CAR-T therapy can be first delivered to the arterial branch that feeds the tumor and immediately, or later, followed by the delivery of the radioembolic beads into the same arterial branch. Thus, the CAR-T therapy is delivered to the tumor and the radioembolic beads proximally occlude the arterial lumen, capturing capture the CAR-T therapy in place, preventing systemic migration of the engineered cells. Radioembolic bead 1 could also deliver therapeutic radiation. In an additional embodiment, one can use the method described in FIGS. 7A and 7B, where a 3rd particle that contains a cellular therapy may be mixed with the radiation emitting particle and the drug-eluting particle.

In some embodiments, the instant embolic beads disclosed herein can be used to treat other indications or cancers with various therapeutics. For example, in some cases, similar to the liver, the lungs are supplied with blood from two distinct supplies from the pulmonary artery and the bronchial artery. As liver tumors are supplied by to the hepatic artery, lung tumors are typically supplied by the bronchial artery. Therefore, much the same as the liver, one can embolize branches of the bronchial artery without damaging the remaining healthy tissue in the lungs. Further still, in some embodiments, the instant disclosure includes applicability in treatment of gliomas in the brain or spinal cord and tumors of the prostate, among others. While the terms “tumor” and “tumor cells” are used herein, such terms can also mean “solid tumors.”

As utilized herein, the terms “comprises” and “comprising” are intended to be construed as being inclusive, not exclusive. As utilized herein, the terms “exemplary”, “example”, and “illustrative”, are intended to mean “serving as an example, instance, or illustration” and should not be construed as indicating, or not indicating, a preferred or advantageous configuration relative to other configurations. As utilized herein, the terms “about”, “generally”, and “approximately” are intended to cover variations that may existing in the upper and lower limits of the ranges of subjective or objective values, such as variations in properties, parameters, sizes, and dimensions. In one non-limiting example, the terms “about”, “generally”, and “approximately” mean at, or plus 10 percent or less, or minus 10 percent or less. In one non-limiting example, the terms “about”, “generally”, and “approximately” mean sufficiently close to be deemed by one of skill in the art in the relevant field to be included. As utilized herein, the term “substantially” refers to the complete or nearly complete extend or degree of an action, characteristic, property, state, structure, item, or result, as would be appreciated by one of skill in the art. For example, an object that is “substantially” circular would mean that the object is either completely a circle to mathematically determinable limits, or nearly a circle as would be recognized or understood by one of skill in the art. The exact allowable degree of deviation from absolute completeness may in some instances depend on the specific context. However, in general, the nearness of completion will be so as to have the same overall result as if absolute and total completion were achieved or obtained. The use of “substantially” is equally applicable when utilized in a negative connotation to refer to the complete or near complete lack of an action, characteristic, property, state, structure, item, or result, as would be appreciated by one of skill in the art.

Numerous modifications and alternative embodiments of the present disclosure will be apparent to those skilled in the art in view of the foregoing description. Accordingly, this description is to be construed as illustrative only and is for the purpose of teaching those skilled in the art the best mode for carrying out the present disclosure. Details of the structure may vary substantially without departing from the spirit of the present disclosure, and exclusive use of all modifications that come within the scope of the appended claims is reserved. Within this specification embodiments have been described in a way which enables a clear and concise specification to be written, but it is intended and will be appreciated that embodiments may be variously combined or separated without parting from the invention. It is intended that the present disclosure be limited only to the extent required by the appended claims and the applicable rules of law.

What is claimed is:

1. An embolic particle for use in treatment of tumor cells, the embolic particle comprising:
an inner core having a volume within which a radioisotope can be accommodated, the inner core including a surface over which a layer can be arranged;
an outer layer having a thickness within which a therapeutic can be accommodated, the outer layer being placed over the surface of the inner core; and
the inner core and outer layer having a combined density sufficient to allow the embolic particle to move with a fluid flow, along a pathway, while the embolic particle includes a dimension that is sufficiently large to engage the pathway to limit fluid flow to the tumor cells.

2. The embolic particle of claim 1, wherein the dimension of the embolic particle is sufficient to substantially block the fluid flow through the pathway to the tumor cells.

3. The embolic particle of claim 2, wherein the embolic particle treats at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

4. The embolic particle of claim 1, wherein the therapeutic is infused in the outer layer.

5. The embolic particle of claim 4, wherein the outer layer releases the therapeutic to the tumor cells while the embolic particle limits the fluid flow to the tumor cells.

6. The embolic particle of claim 5, wherein the embolic particle treats at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

7. The embolic particle of claim 1, wherein the inner core is embedded with a radioisotope.

8. The embolic particle of claim 7, wherein the inner core emits radiation, while the embolic particle limits the fluid flow, to irradiate the tumor cells.

9. The embolic particle of claim 8, wherein the embolic particle treats at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

10. The embolic particle of claim 1, wherein the inner core is embedded with a radioisotope and a therapeutic is impregnated in the outer layer.

11. The embolic particle of claim 10, wherein while the embolic particle limits the fluid flow to the tumor cells,
the inner core emits radiation to kill the tumor cells, and
the outer layer releases the therapeutic to treat remaining tumor cells.

12. The embolic particle of claim 11, wherein the radioisotope includes yttrium-90 (Y-90) or Holmium-166.

13. The embolic particle of claim 11, wherein the embolic particle treats at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.

14. The embolic particle of claim 1, wherein the inner core is formed from at least one of a polymer, ceramic, glass, a glass-ceramic composite.

15. The embolic particle of claim 14, wherein the inner core is sintered with a porosity of about 10% to about 75%.

16. The embolic particle of claim 1, wherein the outer layer forms a uniform shape about the inner core.

17. The embolic particle of claim 1, wherein the outer layer is formed from a polymer.

18. The embolic particle of claim 17, wherein the outer layer is embedded with a radiopaque material.

19. The embolic particle of claim 1, wherein the embolic particle has a substantially spherical shape.

20. The embolic particle of claim 1, wherein the embolic particle has a size of about 15 micrometers-about 1000 micrometers.

21. The embolic particle of claim 1, further comprising at least one additional layer, the at least one additional layer being distinct from the inner core and outer layer.

22. A kit for treatment of tumor cells, the kit comprising,
a plurality of embolic particles, each embolic particle including,
an inner core having a volume within which a radioisotope can be accommodated, the inner core including a surface over which a layer can be arranged;
an outer layer having a thickness within which a therapeutic can be accommodated, the outer layer being placed over the surface of the inner core; and
the inner core and outer layer having a combined density sufficient to allow an embolic particle to move with a fluid flow, along a pathway, while the embolic particle includes a dimension that is sufficiently large to engage the pathway to limit fluid flow to the tumor cells;
a liquid containing the therapeutic and into which the plurality of embolic particles can be soaked to permit the therapeutic to be infused in the outer layer;
a vial to accommodate the liquid.

23. The kit of claim 22, wherein the liquid is disposed in the vial.

24. The kit of claim 22, wherein the therapeutic is infused into the outer layer of the plurality of embolic particles when the plurality of embolic particles are disposed in the liquid.

25. The kit of claim 22, wherein the liquid includes saline.

26. The kit of claim 22, wherein the plurality of embolic particles expand in size when the plurality of embolic particles are disposed in the liquid.

27. The kit of claim 22, wherein the plurality of embolic particles remain the same size when the plurality of embolic particles are disposed in the liquid.

28. A method of manufacturing an embolic particle, the method comprising,
providing an inner core having a volume within which a radioisotope can be accommodated, the inner core including a surface over which a layer can be arranged;
providing a composition within which a therapeutic can be infused, and
arranging the composition over the surface of the inner core to create an outer layer.

29. The method of claim 28, wherein the inner core includes a hollow inner chamber which can accommodate the radioisotope or the therapeutic.

30. The method of claim 28, further comprising,
infusing the radioisotope into the inner core.

31. The method of claim 30, further comprising,
activating the radioisotope such that the embolic particle emits radiation.

32. The method of claim 30, further comprising,
infusing the outer layer with the therapeutic.

33. The method of claim 28, wherein the therapeutic is premixed with the outer layer prior to the arranging step.

34. A method of treatment of tumor cells using embolic particles, the method comprising,
providing a plurality of embolic particles each having an inner core having a volume within which a radioisotope can be accommodated and an outer layer having a thickness within which a therapeutic can be accommodated;
identifying a site of interest that includes the tumor cells to be treated; and
delivering the embolic particles to a lumen and allowing the plurality of embolic particles to move with a fluid flow, along a pathway, to the site of interest such that the embolic particles engage with the pathway to limit fluid flow to the tumor cells.

35. The method of claim 34, further comprising
activating radioisotopes within the inner core.

36. The method of claim 35, further comprising
irradiating the tumor cells at the site of interest.

37. The method of claim 34, further comprising impregnating the therapeutic into respective outer layers of the plurality of embolic particles.

38. The method of claim 37, wherein the impregnating step further comprising,
disposing the plurality of embolic particles into a fluid containing the therapeutic to infuse the therapeutic into at least the outer layer.

39. The method of claim 34, wherein a dimension of respective embolic particles limits movement of the respective embolic particles through the lumen at the site of interest to prevent the plurality of embolic particles from migrating away from the site of interest.

40. The method of claim 34, wherein the delivering step includes delivering the plurality of embolic particles through an arterial branch that feeds the tumor cells.

41. The method of claim 35, the method further comprising,
treating at least one of primary or metastatic tumors of a liver, lungs, the brain or spinal cord, prostate, breast, esophagus, upper aerodigestive tract including the nasopharynx, nasal cavity, oral cavity, oropharynx, hypopharynx, larynx, neck, thyroid, lungs, mediastinum, stomach, small bowel, large bowel, pancreas, spine, kidneys, ureters, bladder, urethra, vagina, uterus, cervix, ovary, lymph nodes, muscles, and bones.
